(19) United States
(12) Patent Application Publication
(10) Pub. No.: US 2021/0186266 A1
(43) Pub. Date: Jun. 24, 2021

(54) ASSESSING A QUALITY OF A COOKING MEDIUM IN A FRYER USING ARTIFICIAL INTELLIGENCE

(71) Applicant: Enodis Corporation, New Port Richey, FL (US)

(72) Inventors: Ramesh B Tirumala, Lutz, FL (US); Himanshu Parikh, Coconut Creek, FL (US)

(73) Assignee: Enodis Corporation

(21) Appl. No.: 17/124,781

(22) Filed: Dec. 17, 2020

Related U.S. Application Data
(60) Provisional application No. 62/949,807, filed on Dec. 18, 2019.

(51) Int. Cl. A47J 37/12; G01N 33/03

(52) U.S. Cl. CPC A47J 37/1266; G01N 33/03

(57) ABSTRACT

There is provided a system and a method for assessing a quality of a cooking medium in a fryer. The system includes a fryer pot, a filtration unit, a conduit, an electronic module, and a processor. The conduit is in fluid communication with the fryer pot for carrying the cooking medium from the fryer pot through the filtration unit back to the fryer pot. The electronic module collects values of a plurality of operating parameters of the fryer, over a period of time. The processor produces an assessment of the quality from an evaluation of the values in accordance with a model of a relationship between the quality and a combination of the operating parameters. There is also provided a storage device that contains instructions for controlling the processor.

### CROSS-REFERENCE TO RELATED APPLICATIONS

The present application is claiming priority of U.S. Provisional Patent Application Ser. No. 62/949,807, filed on Dec. 18, 2019, the content of which is herein incorporated by reference.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### BACKGROUND OF THE DISCLOSURE

### 1. Field of the Disclosure

The present disclosure relates to a system for assessing a quality of a cooking medium in a fryer. In an exemplary embodiment, the system computes and predicts total polar material in cooking oil that is being used in a deep fat fryer, in order to manage oil quality, which in turn results in better food quality, food safety and financial savings for restaurant operators.

### 2. Description of the Related Art

The approaches described in this section are approaches that could be pursued, but not necessarily approaches that have been previously conceived or pursued. Therefore, the approaches described in this section may not be prior art to the claims in this application and are not admitted to be prior art by inclusion in this section.

During use, frying fats undergo chemical deterioration. This leads to the formation of compounds that are more polar than the triacylglycerols of the fat. Collectively these are called total polar material (TPM), and the mass concentration of TPM is used as an indicator of the quality of frying fats.

U.S. Pat. No. 8,497,691 (hereinafter “the '691 patent”), entitled “Oil Quality Sensor and Adapter for Deep Fryers” discloses a system for measuring the state of degradation of cooking oil or fat. In this regard, the '691 patent describes hardware and structural features of such a system, and its entire contents is being herein incorporated by reference.

Existing oil sensing solutions employ some form of hardware sensor or a test strip that is dipped in oil manually and shows a color change. For example, an oil quality sensor (OQS) measures a small capacitance variation in oil to produce a TPM measurement as an indicator of oil quality. The output of such a sensor tends to drift over time, and the sensor requires periodic maintenance or replacement. The sensor is also relatively expensive, e.g., about $1,000.

### SUMMARY OF THE DISCLOSURE

It is an object of the present disclosure to provide a technique for assessing a quality, e.g., TPM, of a cooking medium, e.g., cooking oil, in a fryer that does not have a hardware-based sensor installed therein to measure the quality.

The present document discloses a system and a method for assessing a quality of a cooking medium in a fryer. The system includes a fryer pot, a filtration unit, a conduit, an electronic module, and a processor. The conduit is in fluid communication with the fryer pot for carrying the cooking medium from the fryer pot through the filtration unit back to the fryer pot. The electronic module collects values of a plurality of operating parameters of the fryer, over a period of time. The processor produces an assessment of the quality from an evaluation of the values in accordance with a model of a relationship between the quality and a combination of the operating parameters. The present document also discloses a storage device that contains instructions for controlling the processor.

### BRIEF DESCRIPTION OF THE DRAWINGS

FIG. 1 is a block diagram of a system for assessing a quality of a cooking medium in a fryer, by utilization of a machine learning module.

FIG. 1A is a block diagram of a system that may be used for training the machine learning module in the system of FIG. 1.

FIG. 2 is a block diagram of the machine learning module of the system of FIG. 1.

FIG. 3 is a block diagram of data and information flow in the system of FIG. 1.

FIG. 4 is an illustration of a report that is produced by the system of FIG. 1.

FIG. 5 is an illustration of a table of fryer prediction information, produced by the system of FIG. 1.

FIG. 6 is a set of graphs showing measurements produced using a hardware sensor, and calculations using the system of FIG. 1.

A component or a feature that is common to more than one drawing is indicated with the same reference number in each of the drawings.

### DESCRIPTION OF THE DISCLOSURE

The present disclosure is an innovation around oil quality sensing in deep fat fryers. The innovation is with Artificial Intelligence (AI) technology and Machine Learning (ML) models based on large sets of data collected with fryers running in actual stores. This is a software-based virtual oil quality sensing. The software will send a notification to a user of when to dispose of oil based on TPM calculated with an ML model. This will result in considerable oil savings, e.g., early studies show $3000-4000 per fryer per year. The technique disclosed herein not only calculates a current TPM, but also predicts a future TPM value so that oil disposal can be planned ahead of time.

The technique disclosed herein uses data analytics and machine learning to create a predictive model using data concerning operating parameters such as number of cooks, number of quick filters, oil temperature during idle, and cooking state, coming from one or more fryers operating in one or more real-life stores, and other significant variables. The functionality is to predict TPM values of oil, trend it, and upon reaching a threshold based on oil type, generate a notification to a user to inform the user that it is time to dispose of the oil. This technology replaces the OQS hardware sensor and provides oil savings to users.

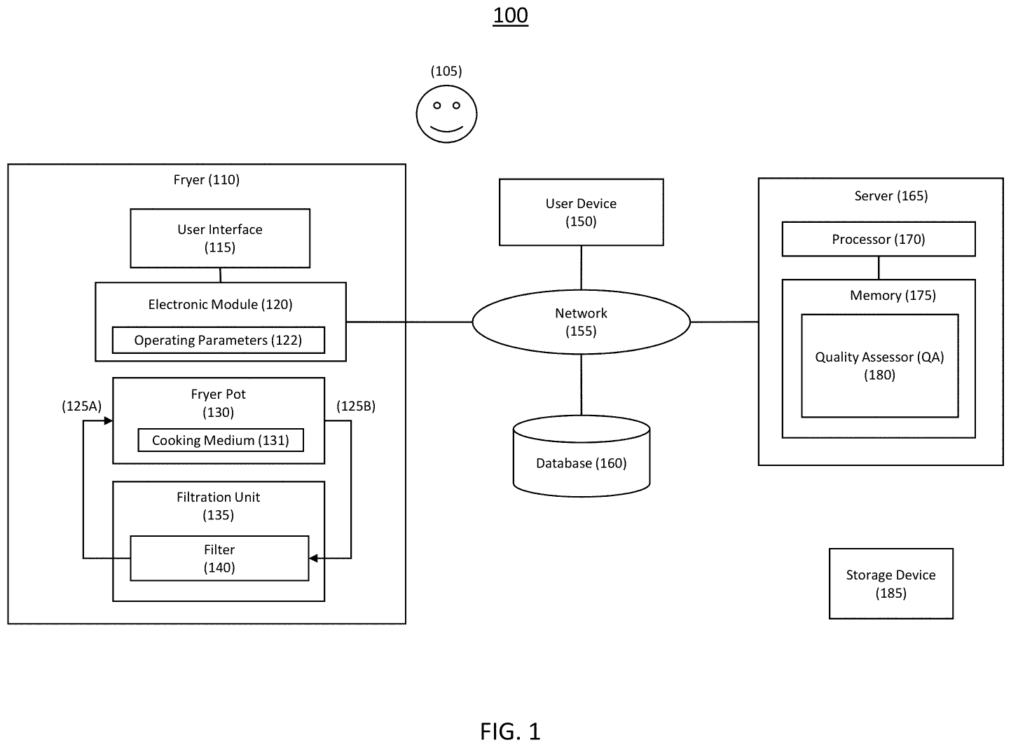

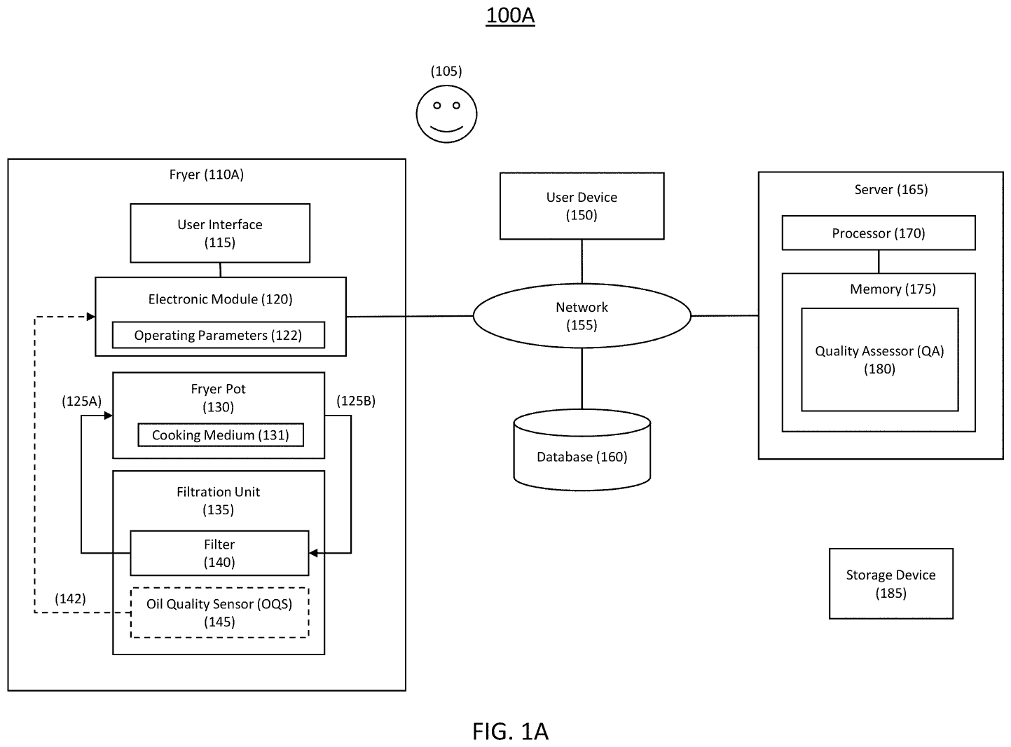

FIG. 1 is a block diagram of a system, namely system 100, for assessing a quality of a cooking medium in a fryer. System 100 includes a fryer 110, a user device 150, a database 160, and a server 165, all of which are communicatively coupled to a network 155.

Network 155 is a data communications network. Network 155 may be a private network or a public network, and may include any or all of (a) a personal area network, e.g., covering a room, (b) a local area network, e.g., covering a building, (c) a campus area network, e.g., covering a campus, (d) a metropolitan area network, e.g., covering a city, (e) a wide area network, e.g., covering an area that links across metropolitan, regional, or national boundaries, (0 the Internet, or (g) a telephone network. Communications are conducted via network 155 by way of electronic signals and optical signals that propagate through a wire or optical fiber, or are transmitted and received wirelessly.

A user 105 operates fryer 110 and user device 150. In practice, user 105 may operate fryer 105, and a second user (not shown) may operate user device 150.

Fryer 110 includes a user interface 115, an electronic module 120, a fryer pot 130, and a filtration unit 135. Filter unit 135 includes a filter 140.

Fryer pot 130, also known as a vat or a frypot, contains a cooking medium 131, e.g., cooking oil, fat or shortening. A conduit formed by conduit sections 125A and 125B is in fluid communication with fryer pot 130 for carrying cooking medium 131 from fryer pot 130, through filtration unit 135, back to fryer pot 130. Thus, cooking medium 131 is circulated from fryer pot 130, through conduit section 125B, filter 140, and conduit section 125A, back to fryer pot 130. Filter 140 removes undesirable material, e.g., food particles, from cooking medium 131.

User interface 115 includes an input device, such as a keyboard, speech recognition subsystem, or gesture recognition subsystem, for enabling user 105 to specify various operating parameters of fryer 110. User interface 115 also includes an output device such as a display or a speech synthesizer and a speaker.

Electronic module 120 controls fryer 110, and collects values of a plurality of operating parameters 122 of fryer 110. Some operating parameters 122 are provided by user 101, via user interface 115, and may include maintenance data like manual filtration and maintenance filtration, change filter pad, oil sensor status (clean oil is back (OIB) sensor). Some operating parameters 122 are inherent in the operation of fryer 110, and obtained by electronic module 120 from other components of fryer 110 during regular operation of fryer 110. There are also fryer systems that automatically perform operations that affect oil quality, for example, automatically maintaining a volume of cooking oil in a fryer pot, which is referred to as automatic top-off. U.S. Pat. No. 8,627,763, the entire content of which is being herein incorporated by reference, discloses a system for automatic top-off for deep fat fryers. Operating parameters 122 include:

(a) number of cooks per day between disposals;
(b) number of quick filters per day between disposals;
(c) number of clean filters per day between disposals;
(d) time spent in the specific machine status-temperature pair per day between disposals;
(e) number of specific temperatures drops per day between disposals; and
(f) difference of actual and planned cooking time per day between disposals.
(g) high temperature-idle;
(h) low temperature-cooking;
(i) medium temperature-cooking;
(j) high temperature-cooking;
(k) high temperature-drop;
(l) type of cooking medium;
(m) type and quantity of product cooked;
(n) pan present;
(o) change filter pad;
(p) actual sensor error status;
(q) indication that fresh cooking medium has been brought in by means other than regular practice;
(r) time in a cooking state;
(s) oil added during an automatic top-off; and
(t) information about automatic operations that affect the quality of the cooking medium.

Knowledge of the pan present, i.e., item (n), above, improves model performance, as it ensured oil disposal/change happened physically as oil drained to pan, during which pan is removed and inserted.

Knowledge of the change filter pad, i.e., item (o), above, improves model performance, as it ensured oil disposal/change happened.

Knowledge of the actual sensor error status, i.e., item (p), above, helps during training of a model to ignore sensor values when there was information indicating that the hardware sensor was in error.

Information about automatic operations that affect the quality of the cooking medium includes information about automatic top-off or other methods that bring in fresh oil, or automatic change of fryer state such as idle, standby or cooking.

User device 150 is a device such as a computer or a smart phone, through which user 101 can receive information from, or send information to, server 165, and which includes a display on which the information can be presented.

Server 165 is a computer that includes a processor 170, and a memory 175 that is operationally coupled to processor 170. Although server 165 is represented herein as a standalone device, it is not limited to such, but instead can be coupled to other devices (not shown) in a distributed processing system.

Processor 170 is an electronic device configured of logic circuitry that responds to and executes instructions.

Memory 175 is a tangible, non-transitory, computer-readable storage device encoded with a computer program. In this regard, memory 175 stores data and instructions, i.e., program code, that are readable and executable by processor 170 for controlling operations of processor 170. Memory 175 may be implemented in a random access memory (RAM), a hard drive, a read only memory (ROM), or a combination thereof. One of the components of memory 175 is a program module, namely quality assessor (QA) 180, which contains instructions for controlling processor 170 to execute operations described herein.

The term “module” is used herein to denote a functional operation that may be embodied either as a stand-alone component or as an integrated configuration of a plurality of subordinate components. Thus, QA 180 may be implemented as a single module or as a plurality of modules that operate in cooperation with one another. Moreover, although QA 180 is described herein as being installed in memory 175, and therefore being implemented in software, it could be implemented in any of hardware (e.g., electronic circuitry), firmware, software, or a combination thereof.

Processor 170 outputs, to user interface 115 and/or user device 150, a result of an execution of the methods described herein.

While QA 180 is indicated as being already loaded into memory 175, it may be configured on a storage device 185 for subsequent loading into memory 175. Storage device 185 is a tangible, non-transitory, computer-readable storage device that stores QA 180 thereon. Examples of storage device 185 include (a) a compact disk, (b) a magnetic tape, (c) a read only memory, (d) an optical storage medium, (e) a hard drive, (f) a memory unit consisting of multiple parallel hard drives, (g) a universal serial bus (USB) flash drive, (h) a random access memory, and (i) an electronic storage device coupled to server 165 via network 155.

Database 160 holds data that is utilized by QA 180. Although database 160 is represented herein as a standalone device, it is not limited to such, but instead can be coupled to other devices (not shown) in a distributed database system. Database 160 could also be located in close proximity to server 165, rather than being located remotely from server 165.

Electronic module 120 collects values of operating parameters 122 of fryer 110, over a period of time, and sends the values to processor 170. The period of time depends on the nature of the quality that is being assessed, but would be of a duration that is adequate to assess the quality, and in practice, would typically be seconds, minutes, hours, days, or weeks. Processor 170, pursuant to instructions in QA 180, produces an assessment of a quality of cooking medium 131 from an evaluation of the values, in accordance with a model of a relationship between the quality and a combination of operating parameters 122.

Although processor 170, memory 175, and QA 180 are shown as being embodied in server 165, they can, instead, be embodied in fryer 110. Database 160 can also be embodied in fryer 110. As such, fryer 100 can be configured as a stand-alone system.

Because the oil type of cooking medium 131, or other operational factors, may be different for different fryers, a training mode may be executed, for an initial training period (short 90 days or so) to train QA 180.

FIG. 1A is a block diagram of a system, namely system 100A, that may be used for training QA 180. System 100A is similar to system 100. However, system 100A includes a fryer 110A that includes an optional component, namely an oil quality sensor (OQS) 145, that is not included in fryer 110. Since OQS 145 is optional, it is being represented with a dashed line. When OQS 145 is installed, it is located in or near filtration unit 135. OQS 145 is a hardware device that measures a property of cooking medium 131, e.g., capacitance, as cooking medium 131 circulates through filtration unit 135. Thus, OQS 145 could be used to detect the presence of extraneous material, e.g., TPM, in cooking medium 131. OQS 145 reports the measured property to electronic module 120 via a connector 142. The measured property would be among operating parameters 122 that electronic module 120 obtains and reports to QA 180, and that QA 180 would consider when executing a training mode to develop quality models. After the training period, OQS 145 can be removed from fryer 110A. OQS 145 will no longer be needed, as QA 180 will calculate and predict the TPM.

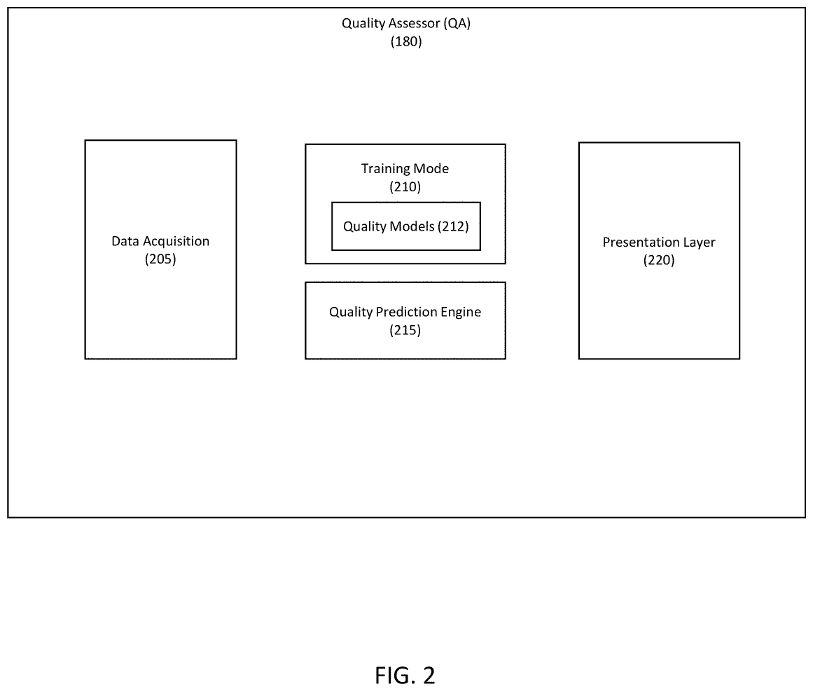

FIG. 2 is a block diagram of QA 180. QA 180 is a machine learning module and includes subordinate modules designated as data acquisition 205, training mode 210, quality prediction engine 215, and presentation layer 220. For convenience, QA 180 is described herein as performing certain operations, but in practice, the operations are actually performed by processor 170.

Data acquisition 205 communicates with electronic module 120 to obtain operating parameters 122.

Training mode 210 evaluates values of operating parameters 122, and based thereon, develops quality models 212. Quality models 212 are thus, machine learning models, for example, general additive models, or deep learning models based on a neural network.

Quality models 212 are models of relationships between (i) one or more qualities of cooking medium 131, and (ii) one or more combinations of operating parameters 122. In practice, system 100 may include a plurality of fryers that are configured similarly to fryer 110. Server 165 may therefore receive values of operating parameters from the plurality of fryers, and quality models 212 may be developed based on historical values of operating parameters for the plurality of fryers. Quality models 212 and the data that is used to develop them may be stored in database 160.

Quality prediction engine 215 utilizes quality models 212 to assess one or more qualities of cooking medium 131. Quality prediction engine 215 produces an assessment of a quality from an evaluation of values of operating parameters 122 in accordance with a model of a relationship, from quality models 212, between the quality and a combination of operating parameters 122. For example, the quality may be indicative of a characteristic of cooking medium 131, e.g., the purity of cooking medium 131, and the assessment may quantify an aspect of the characteristic, e.g., indicate a quantity of TPM in cooking medium 131. Quality prediction engine 215 may issue a recommendation of a maintenance action based on the assessment, e.g., to dispose of cooking medium 131. The recommendation may include a prediction of a future time to dispose of cooking medium 131, e.g., predicting that cooking medium 131 should be disposed of in two days from today.

Presentation layer 220 communicates with user interface 115 and/or user device 150, to report a result of an execution of quality prediction engine 215.

Thus, pursuant to instructions in QA 180, processor 170 performs a method for assessing a quality of a cooking medium in a fryer. The method includes (a) receiving values of a plurality of operating parameters of the fryer that have been collected over a period of time, and (b) producing an assessment of the quality from an evaluation of the values in accordance with a model of a relationship between the quality and a combination of the operating parameters.

AI is a technology used to create hardware and/or software solutions for solving real world engineering problems. In order to create usable solutions, different disciplines are involved, for example, algorithm theory, statistics, software engineering, computer science/engineering, mathematics, control theory, graph theory, physics, computer graphics, image processing, etc. When developing QA 180, we started with a two/three variable statistical model, which provided satisfactory results, but we migrated to a more complex neural network-based model for better model performance and accuracy.

A neural network is a type of artificial intelligence that is inspired by how a brain works, and is fashioned after a human brain. A dendroid in a human brain is connected to a nucleus, and the nucleus is connected to an axon. Inputs are like dendroids, a nucleus is where the complex calculations occur (e.g., weighed sum, activation function), and the axon is the output.

The way a neural network learns is more complex, as compared to other traditional classification or regression models. A neural network model has many internal variables, and the relationships between input variables and output may go through multiple internal layers. Neural networks have higher accuracy as compared to other supervised learning algorithms.

QA 180 is an AI engine that uses a neural network. The neural network includes hidden layers that can vary, and will vary as the neural network learns. In this regard, QA 180 utilizes AI computational libraries to develop quality models 212, which evolve, and improve as they evolve. QA 180 takes input data and separates it into training and test/validation sets in a certain meaningful ratio. The ratios can be programmed, e.g., typically 80% and 20%, and after this step, data is normalized so that they fall in between a minimum and maximum range needed for these type of computations. These are then passed into one or more computational library/methods that do the subsequent steps of model fitting, predicting, and visualization with plotting, etc. In system 100, one result is a TPM number. Once the model is developed, when new data from fryer 110 is fed into the model and processed/consumed by the model, it generates/predicts an output TPM value. This is done based on a pattern, i.e., in the hidden layers, that was developed over a large set of data, and the neural network represents this pattern. As system 100 collects data, the model continuously improves, and the time for data collection may extend over a long period for improved accuracy.

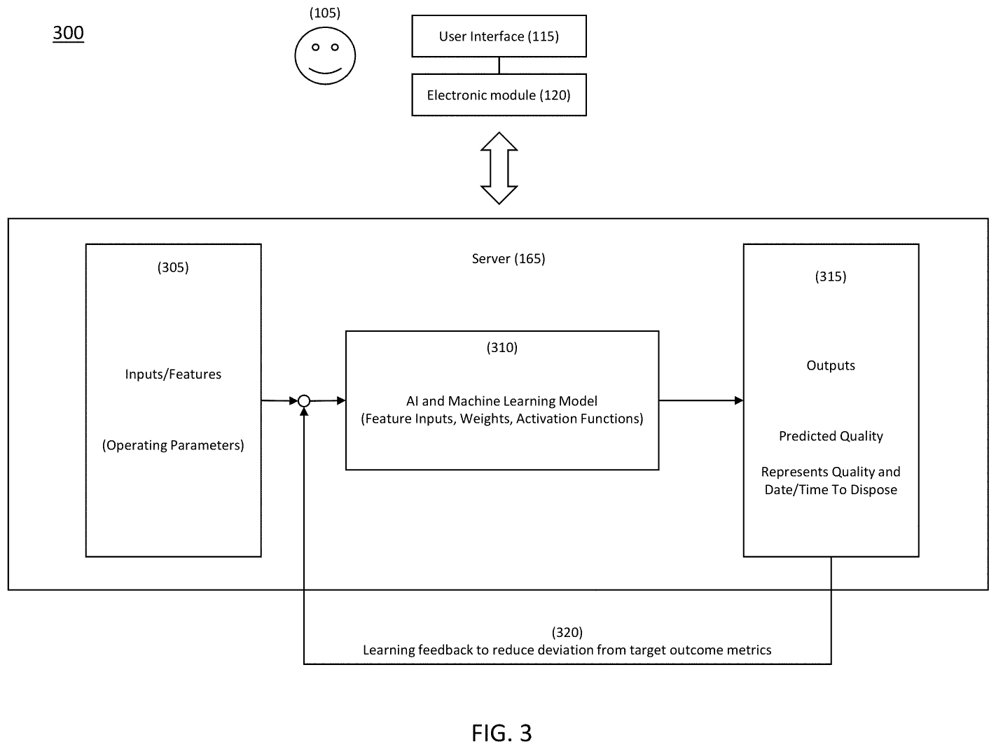

FIG. 3 is a block diagram of data and information flow 300, in system 100. Electronic module 120 obtains some operating parameters 122 from user 105 via user interface 115, and some operating parameters 122 from other components of fryer 110 during regular operation of fryer 110. Electronic module 120 sends operating parameters 122 to QA 180.

In block 305, QA 180 receives operating parameters 122 as feature inputs.

In block 310, QA 180 utilizes AI processes and a machine learning model, and considers the feature inputs, and also considers weights, and activation functions. Weights indicate importance we give to certain data inputs, some have higher weight (filters, cooks, type of product, oil temperature) compared to others in the prediction model. Activation functions are used in neural networks. They help provide needed non-linearity in models, as the relationships among inputs to the output is complex. Examples are sigmoid, Tanh, ReLu functions.

In block 315, QA 180 generates outputs such as a predicted quality of cooking medium 131, and information that represents the quality and a predicted date/time to dispose of cooking medium 131, and sends outputs to (i) user interface 115 via electronic module 120, and (ii) user device 150.

Information flow 300 also includes a feedback loop 320, which includes learning feedback to reduce deviation from target outcome metrics. This is a supervised learning model where there is a training set of data and validation/test data. The model evolves with time as new features/inputs are added, to improve accuracy, as part of training data. The new feature for example could be an operational parameter that was not previously known when the initial model was developed. This new feature is added when the target accuracy is not reached and hence is represented as a feedback loop. Thus, QA 180 receives feedback concerning operation of fryer 110, and modifies quality models 212 based on the feedback. Since QA 180 is a machine learning system, as more data is accumulated for quality models 212, quality models 212 evolve and are improved over time, and QA 180 performs better over time.

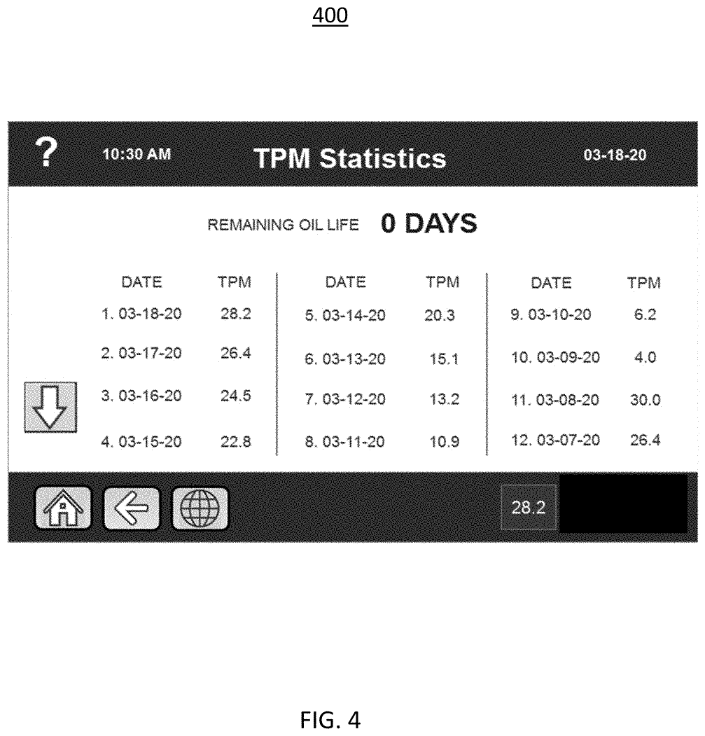

FIG. 4 is an illustration of an exemplary report 400 that is produced by QA 180 for presentation on either or both of user interface 115 and user device 150. Report 400 has a report date of Mar. 18, 2020, and shows TPM for cooking oil for dates leading up to Mar. 18, 2020. For example:

on Mar. 7, 2020, the TPM was 26.4;

on Mar. 8, 2020, the TPM was 30.0; and

on Mar. 9, 2020, the TPM was 4.0.

Since the TPM on Mar. 9, 2020 is less than the TPM on Mar. 8, 2020, the cooking oil was changed sometime between the assessments generated on Mar. 8, 2020 and Mar. 9, 2020. Assume that the threshold of acceptable TPM is 24. The TPM values show a rising trend from the time fresh oil is brought in (between Mar. 8, 2020 and Mar. 9, 2020) to the time it exceeds the threshold of 24 (between Mar. 15, 2020 and Mar. 16, 2020), resulting in showing oil has to be changed now (on Mar. 18, 2020) and therefore Remaining Oil Life is 0 days as shown on the top line. Actually, since the threshold was exceeded sometime between Mar. 15, 2020 and Mar. 16, 2020, and the report is dated Mar. 18, 2020, the oil change is past due.

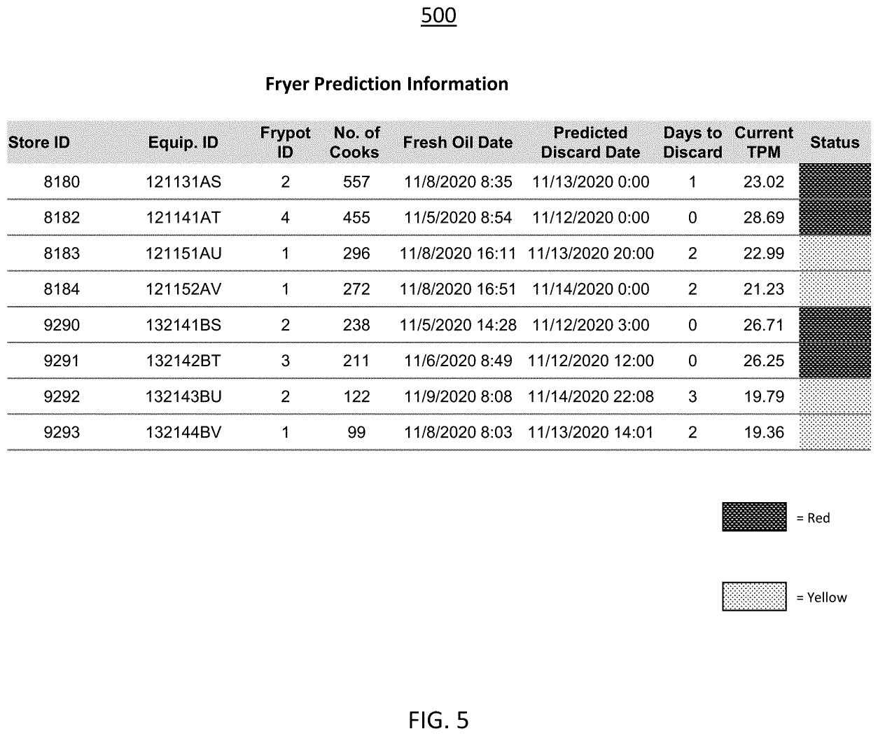

FIG. 5 is an illustration of a table 500 of fryer prediction information. As mentioned above, system 100 may include a plurality of fryers that are configured similarly to fryer 110. The fryers send operation and maintenance data to server 165, which runs QA 180 for oil disposal prediction. Based on the collected data, and the associated operating parameters that are used in quality models 212, QA 180 produces an assessment that includes Fresh Oil Date, Predicted Disposal Date, Days to Dispose, Current TPM and status. This assessment is presented to user device 150 to help operators proactively manage their fryer and vat oil condition.

Table 500 shows for each frypot, in a plurality of stores, a prediction date for oil discard along with days to discard with status of Red to alert a user that the time has expired on some of the frypots to discard the oil. A status of Yellow indicates that there are few days remaining to discard, giving time for users to plan work ahead of time.

The technique disclosed herein is based on data (e.g., number of cooks, number of quick filters, oil temperature profile, etc.) collected from fryers operating in a real-life situation, and then using this data and looking at highly correlated variables to predict the oil quality (TPM), and sending an alert to a user, via user interface 115 and/or user device 150, to change the frypot oil. An application can be installed on user device 150 to provide information, from QA 180, about all the fryers that are approaching oil disposal time or past disposal, where multiple fryers are associated with a user, a chart of how the TPM is trending in every fryer, when the last oil change was made, cooks since last oil change, and other useful metrics.

Thus, processor 170, pursuant to instructions in QA 180, computes TPM based on a trained model, i.e., one or more of quality models 212, and predicts the date/time to discard cooking medium 131, e.g., cooking oil. QA 180 uses supervised machine learning. A training dataset is used to build a current training model. The model is deployed to take in new data (significant variables) and predict TPM value. This is termed an inference model. The inference model can be deployed locally at the edge of or in the cloud for each instance of a fryer.

QA 180 may be regarded as a virtual OQS. Benefits of QA 180 include:
(a) avoiding a hardware-based sensor which is bulky, expensive, and needs maintenance;
(b) oil savings by properly disposing or avoid disposing based on true condition of oil usage;
(c) enhanced food quality of cooked product as the oil is maintained properly by monitoring and learning the degradation; and
(d) improved food safety as the proper time to oil disposal is notified to a user.

Having a software-based ML solution helps predict TPM even if a hardware OQS is present, but malfunctioning. In addition, the prediction aspect of QA 180 informs user 105 well ahead of time when to dispose oil so that user 105 can better plan the activity of oil disposal and bringing in fresh oil.

Thus, system 100, in comparison to prior art systems, provides reduced costs in the form of:

(a) less hardware, or at least no additional hardware, e.g., no additional sensor;
(b) reduced support and maintenance costs for servicing part in the field; and
(c) oil savings.

While contemplating system 100, the present inventor recognized that the following factors contribute to the degradation of oil quality:

(a) proper design, construction and maintenance of equipment;
(b) proper cleaning of equipment;
(c) moisture content of food; and
(d) amount of food that is cooked.

Most of these factors are not readily available from the dataset and they need to be indirectly inferred. Considering these factors, several potential explanatory variables have been investigated for this analysis. These variables are the number of cooks per day, number of quick filters per day, and number of clean filters per day along with the temperature profile of the oil in the pot/vat.

In order to model the amount of food that is cooked, the present inventor proposed to measure the drop in temperature at the beginning of each cook. For this variable we can consider two levels; namely, high drop (drop to less than 330 F) and low drop (drop to above 330 F). Moreover, we consider the difference between the actual cooking time and planned cooking time as another contributing factor to the degradation of oil.

A large (over a year) connected fryer dataset was collected and analyzed. Several supervised machine learning models were evaluated and concluded that the general additive model (GAM), as shown below, was found to be very effective. This model was derived by studying the effect of several variables, including:

(a) cumulative number of cooks per day between disposals;
(b) cumulative number of quick filters per day between disposals;
(c) cumulative number of clean filters per day between disposals;
(d) cumulative time spent in the specific machine status-temperature pair per day between disposals;
(e) cumulative number of specific temperatures drops per day between disposals; and
(f) cumulative difference of actual and planned cooking time per day between disposals.

Based on Bayesian Information Criterion (BIC), significant variables were found to be:

(a) number of quick filters;
(b) number of cooks;
(c) high temperature-idle;
(d) low temperature-cooking;
(e) medium temperature-cooking;
(f) high temperature-cooking; and
(g) high temperature-drop.

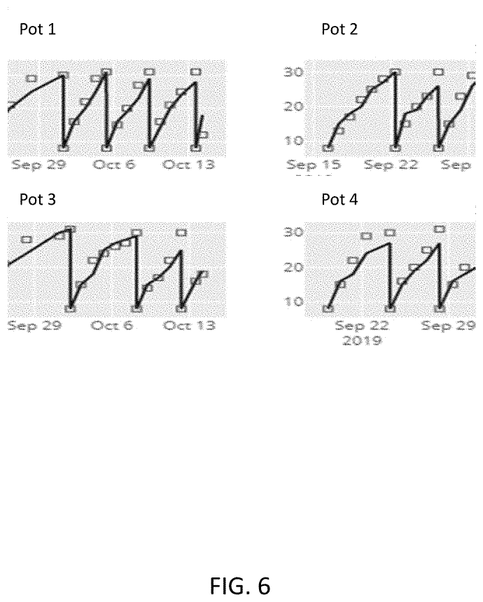

FIG. 6 is a set of graphs showing measurements of TPM produced using a hardware sensor, and calculations of TPM produced in accordance with an AI/ML model as would be used by QA 180. The graphs are for four pots, i.e., a 4-vat fryer. In the graphs, rectangles represent hardware sensor data, and solid curves represent TPM values from the AI/ML model. This illustrates the accuracy of the AI/ML model as compared with the hardware sensor.

To review, the present document discloses a system, i.e., system 100, for assessing a quality of a cooking medium in a fryer. The system includes a fryer pot, a filtration unit, a conduit, and an electronic module. The conduit is in fluid communication with the fryer pot for carrying the cooking medium from the fryer pot through the filtration unit back to the fryer pot. The electronic module collects values of a plurality of operating parameters of the fryer, over a period of time. The processor produces an assessment of the quality from an evaluation of the values in accordance with a model of a relationship between the quality and a combination of the operating parameters.

The present document also discloses a method for assessing a quality of a cooking medium in a fryer. In system 100, the method is performed by processor 170 and includes (a) receiving values of a plurality of operating parameters of the fryer that have been collected over a period of time, and (b) producing an assessment of the quality from an evaluation of the values in accordance with a model of a relationship between the quality and a combination of the operating parameters.

The present document also discloses a non-transitory storage device, i.e., storage device 185, that is encoded with instructions that are readable by a processor, to control the processor to perform operations of (a) receiving values of a plurality of operating parameters of the fryer that have been collected over a period of time, and (b) producing an assessment of the quality from an evaluation of the values in accordance with a model of a relationship between the quality and a combination of the operating parameters.

The techniques described herein are exemplary, and should not be construed as implying any particular limitation on the present disclosure. It should be understood that various alternatives, combinations, and modifications could be devised by those skilled in the art. For example, steps associated with the processes described herein can be performed in any order, unless otherwise specified or dictated by the steps themselves. The present disclosure is intended to embrace all such alternatives, modifications and variances that fall within the scope of the appended claims.

The terms “comprises” or “comprising” are to be interpreted as specifying the presence of the stated features, integers, steps, or components, but not precluding the presence of one or more other features, integers, steps or components or groups thereof. The terms “a” and “an” are indefinite articles, and as such, do not preclude embodiments having pluralities of articles.

What is claimed is:

1. A system for assessing a quality of a cooking medium in a fryer, said system comprising:
a fryer pot;
a filtration unit;
a conduit in fluid communication with said fryer pot for carrying said cooking medium from said fryer pot through said filtration unit back to said fryer pot;
an electronic module that collects values of a plurality of operating parameters of said fryer, over a period of time; and
a processor that produces an assessment of said quality from an evaluation of said values in accordance with a model of a relationship between said quality and a combination of said operating parameters.

2. The system of claim 1, wherein said assessment indicates a quantity of total polar material in said cooking medium.

3. The system of claim 2, wherein said cooking medium is cooking oil.

4. The system of claim 1, wherein said processor issues a recommendation of a maintenance action based on said assessment.

5. The system of claim 4, wherein said recommendation includes a prediction of a future time to dispose of said cooking medium.

6. The system of claim 1, wherein said operating parameter is selected from the group consisting of:
(a) number of cooks per day between disposals;
(b) number of quick filters per day between disposals;
(c) number of clean filters per day between disposals;
(d) time spent in the specific machine status-temperature pair per day between disposals;
(e) number of specific temperatures drops per day between disposals; and
(f) difference of actual and planned cooking time per day between disposals.
(g) high temperature-idle;
(h) low temperature-cooking;
(i) medium temperature-cooking;
(j) high temperature-cooking;
(k) high temperature-drop;
(l) type of cooking medium;
(m) type and quantity of product cooked;
(n) pan present;
(o) change filter pad;
(p) actual sensor error status;
(q) indication that fresh cooking medium has been brought in by means other than regular practice;
(r) time in a cooking state;
(s) oil added during an automatic top-off; and
(t) information about automatic operations that affect the quality of the cooking medium.

7. The system of claim 1, wherein said model is based on historical values of said plurality of operating parameters for a plurality of fryers.

8. The system of claim 1, wherein said model is developed by a machine learning module during execution of a training mode.

9. The system of claim 8, wherein said machine learning module receives feedback concerning operation of said fryer, and modifies said model based on said feedback.

10. The system of claim 8, wherein said model is selected from the group consisting of:
(a) a general additive model; and
(b) a deep learning model based on a neural network.

11. A method for assessing a quality of a cooking medium in a fryer, said method comprising:
receiving values of a plurality of operating parameters of said fryer that have been collected over a period of time; and
producing an assessment of said quality from an evaluation of said values in accordance with a model of a relationship between said quality and a combination of said operating parameters.

12. The method of claim 11, wherein said assessment indicates a quantity of total polar material in said cooking medium.

13. The method of claim 12, wherein said cooking medium is cooking oil.

14. The method of claim 11, further comprising, issuing a recommendation of a maintenance action based on said assessment.

15. The method of claim 14, wherein said recommendation includes a prediction of a future time to dispose of said cooking medium.

16. The method of claim 11, wherein said operating parameter is selected from the group consisting of:
(a) number of cooks per day between disposals;
(b) number of quick filters per day between disposals;
(c) number of clean filters per day between disposals;
(d) time spent in the specific machine status-temperature pair per day between disposals;
(e) number of specific temperatures drops per day between disposals; and
(f) difference of actual and planned cooking time per day between disposals.
(g) high temperature-idle;
(h) low temperature-cooking;
(i) medium temperature-cooking;
(j) high temperature-cooking;
(k) high temperature-drop;
(l) type of cooking medium;
(m) type and quantity of product cooked;
(n) pan present;
(o) change filter pad;
(p) actual sensor error status;
(q) indication that fresh cooking medium has been brought in by means other than regular practice;
(r) time in a cooking state;
(s) oil added during an automatic top-off; and
(t) information about automatic operations that affect the quality of the cooking medium.

17. The method of claim 11, wherein said model is based on historical values of said plurality of operating parameters for a plurality of fryers.

18. The method of claim 11, wherein said model is developed by a machine learning module during execution of a training mode.

19. The method of claim 18, wherein said machine learning module receives feedback concerning operation of said fryer, and modifies said model based on said feedback.

20. The method of claim 18, wherein said model is selected from the group consisting of:
(a) a general additive model; and
(b) a deep learning model based on a neural network.

21. A storage device that is non-transitory and comprises instructions that are readable by a processor, to assess a quality of a cooking medium in a fryer by causing said processor to perform operations of:
receiving values of a plurality of operating parameters of said fryer that have been collected over a period of time; and
producing an assessment of said quality from an evaluation of said values in accordance with a model of a relationship between said quality and a combination of said operating parameters.

22. The storage device of claim 21, wherein said assessment indicates a quantity of total polar material in said cooking medium.

23. The storage device of claim 22, wherein said cooking medium is cooking oil.

24. The storage device of claim 21, wherein said operations also include issuing a recommendation of a maintenance action based on said assessment.

25. The storage device of claim 24, wherein said recommendation includes a prediction of a future time to dispose of said cooking medium.

26. The storage device of claim 21, wherein said operating parameter is selected from the group consisting of:
(a) number of cooks per day between disposals;
(b) number of quick filters per day between disposals;
(c) number of clean filters per day between disposals;
(d) time spent in the specific machine status-temperature pair per day between disposals;
(e) number of specific temperatures drops per day between disposals; and
(f) difference of actual and planned cooking time per day between disposals.
(g) high temperature-idle;
(h) low temperature-cooking;
(i) medium temperature-cooking;
(j) high temperature-cooking;
(k) high temperature-drop;
(l) type of cooking medium;
(m) type and quantity of product cooked;
(n) pan present;
(o) change filter pad;
(p) actual sensor error status;
(q) indication that fresh cooking medium has been brought in by means other than regular practice;
(r) time in a cooking state;
(s) oil added during an automatic top-off; and
(t) information about automatic operations that affect the quality of the cooking medium.

27. The storage device of claim 21, wherein said model is based on historical values of said plurality of operating parameters for a plurality of fryers.

28. The storage device of claim 21, wherein said model is developed by a machine learning module during execution of a training mode.

29. The storage device of claim 28, wherein said machine learning module receives feedback concerning operation of said fryer, and modifies said model based on said feedback.

30. The storage device of claim 28, wherein said model is selected from the group consisting of:
(a) a general additive model; and
(b) a deep learning model based on a neural network.
